(19) United States
(12) Patent Application Publication
(10) Pub. No.: US 2002/0192165 A1
(43) Pub. Date: Dec. 19, 2002

(54) Aerosol compositions comprising a polysaccharide grafted with a polysiloxane

(75) Inventors: Franck Giroud, Clichy (FR); Christine Dupuis, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(73) Assignee: L'OREAL

(21) Appl. No.: 10/094,728

(22) Filed: Mar. 12, 2002

(30) Foreign Application Priority Data
Mar. 13, 2001 (FR) 01 03407

(51) Int. Cl. A61K 9/00; A61K 7/06; A61K 7/11

(52) U.S. Cl. 424/47; 424/70.12

(57) ABSTRACT

An aerosol composition comprising (a) at least one polymer comprising a polysaccharide skeleton grafted with at least one group comprising at least one polysiloxane, and (b) a propellant comprising dimethyl ether. The invention also relates to a method of making the composition, an aerosol device containing the composition, and a cosmetic hair process, for fixing and/or holding the hairstyle by using this aerosol composition.

The invention relates to an aerosol composition comprising (a) a liquid phase comprising at least one fixing material in a suitable solvent, the at least one fixing material comprising at least one polymer comprising a polysaccharide skeleton grafted with at least one group comprising at least one polysiloxane, and (b) a propellant comprising dimethyl ether. The invention also relates to a cosmetic hair process, such as a process for fixing and/or holding the hairstyle by using this aerosol composition, as well as to an aerosol device comprising this composition.

Among the hair products for shaping and/or holding the hairstyle that are most common on the cosmetics market, mention may be made of spray compositions including a solution, which is usually alcoholic or aqueous, and of one or more materials, generally polymer resins, whose function is to form welds between hairs, also known as fixing materials, mixed with various cosmetic adjuvants. This solution is generally packaged either in a suitable aerosol container pressurized by using a propellant, or in a pump-dispenser bottle.

Fixing materials are generally fixing polymers, that is to say film-forming polymers that are soluble in water and in alcohol, such as polyvinylpyrrolidone, vinylpyrrolidone/vinyl acetate copolymers, described in U.S. Pat. Nos. 3,929,735 and 3,770,683, vinyl acetate/crotonic acid copolymers, and anionic or amphoteric acrylic resins. These materials can readily achieve the fixing effect, but after brushing or combing, the hair may have, under the usual lacquering conditions, a stiff appearance and a coarse or even sticky feel, and also a powdered appearance after drying.

Moreover, the quality of the spraying obtained by using an aerosol device is occasionally insufficient. With certain cosmetic compositions it can be difficult, for example, to adjust the droplet size or the spray flow rate as desired.

These drawbacks can be linked to at least one of several parameters, including the nature of the fixing polymer(s), the nature of the welds, and the composition of the propellant gases. To overcome these drawbacks, it is thus possible to act on several parameters without, however, reducing the desired fixing effect.

The inventors have now found that by selecting both the fixing polymer and the propellant gas, it is possible to overcome at least one of the various problems mentioned above. A spray of high quality in terms of diffusion and droplet size, cosmetic properties and powdering, can be obtained.

One subject of the invention is an aerosol composition comprising (a) a liquid phase comprising at least one fixing material in a suitable solvent, the at least one fixing material comprising at least one polymer comprising a polysaccharide skeleton grafted with at least one group comprising at least one polysiloxane, and (b) a propellant comprising dimethyl ether.

Another subject of the invention comprises a method of preparing a composition, comprising dissolving, in an organic solvent, (a) a polysaccharide containing carboxyl groups, and (b) a polysiloxane containing an epoxy end group and corresponding to formula (I), defined below, for a time and under conditions sufficient to obtain a polysaccharide and polysiloxane mixture. The method further comprises heating the mixture to a temperature of between 60 and 200° C. inclusive; reacting the carboxyl groups of the polysaccharide with the epoxy groups of the polysiloxanes for a time and under conditions sufficient to form a polymer comprising a polysaccharide skeleton grafted with at least one group comprising at least one polysiloxane; and adding a propellant comprising dimethyl ether to the grafted polymer to prepare said composition.

Another subject of the invention relates to a cosmetic hair process for fixing and/or holding a hairstyle, comprising applying to hair, an effective amount of a composition comprising, in a cosmetically acceptable medium: (a) at least one polymer comprising a polysaccharide skeleton grafted with at least one group comprising at least one polysiloxane, and (b) a propellant comprising dimethyl ether.

Another subject of the invention relates to the use of this composition for holding and/or shaping the hairstyle, comprising applying to hair, an effective amount of a composition comprising, in a cosmetically acceptable medium: (a) at least one polymer comprising a polysaccharide skeleton grafted with at least one group comprising at least one polysiloxane, and (b) a propellant comprising dimethyl ether.

Yet another subject of the invention relates to an aerosol device comprising a container containing an aerosol composition, the composition comprising (a) a liquid phase comprising at least one fixing material in a suitable solvent, the at least one fixing material comprising at least one polymer comprising a polysaccharide skeleton grafted with at least one group comprising at least one polysiloxane, and (b) a propellant comprising dimethyl ether.

The at least one polymer comprising a polysaccharide skeleton grafted with at least one group comprising at least one polysiloxane, in accordance with the present invention, comprises a principal chain formed from polysaccharide(s), onto which is grafted, and also optionally attached to at least one of its ends, at least one group comprising a polysiloxane.

The at least one polymer comprising a polysaccharide skeleton grafted with at least one group comprising at least one polysiloxane, in accordance with the present invention, may be obtained according to any means known to those skilled in the art, such as a reaction between: (i) a starting polysiloxane macromer functionalized on the polysiloxane chain; and (ii) at least one polysaccharide, which can be functionalized with a group that is capable of reacting with the functional group(s) borne by the said silicone to form a covalent bond.

Exemplary polysaccharide grafted polymers are described in U.S. Pat. No. 6,066,727, by the company Shin-Etsu, the disclosure of which is specifically incorporated by reference herein. These are copolymers obtained by reaction between a polysaccharide containing carboxyl groups and a polysiloxane containing an epoxy end group, in an organic solvent, optionally in the presence of a catalyst.

In one embodiment, the process for preparing these polymers comprises:

(a) dissolving, in an organic solvent, (a) a polysaccharide containing carboxyl groups, and (b) a polysiloxane containing an epoxy end group, and corresponding to formula (I) of a polysiloxane below:

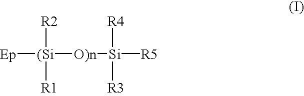

in which

n is an integer between 3 and 500 inclusive,

R₁, R₂, R₃, R₄ and R₅ are chosen, independently of each other, from monovalent C₁ to C₁₀ hydrocarbons and monovalent C₁ to C₁₀ halohydrocarbons, and

Ep is 2-(3,4-epoxycyclohexyl)ethyl; and

(b) heating this mixture of polysaccharides and polysiloxanes to a temperature between 60 and 200° C. inclusive, so as to make the carboxyl groups of the polysaccharides react with the epoxy groups of the polysiloxanes.

Exemplary polysaccharides suitable for carrying out step (a) of this process, include polysaccharides containing at least one group chosen from carboxylic, benzoyl and succinoyl groups, such as hydroxypropylmethylcellulose phthalate and hydroxypropylmethylcellulose acetate succinate, and also include carboxymethylethylcellulose, and the polysaccharide pullulan acetate phthalate.

R₁, R₂, R₃, R₄ and R₅ can be chosen, independently of each other, from methyl, ethyl, propyl and butyl groups, C₃ to C8 cycloalkyl groups such as cyclopentyl and cyclohexyl radicals, aryl groups such as phenyl and tolyl radicals, C₃ to C₈ aralkyl groups, such as benzyl and phenethyl radicals, and alkenyl groups, such as vinyl and allyl groups. These monovalent hydrocarbon radicals may optionally be totally and partially substituted, such as with a halogen atom, for example, with chloromethyl and 3,3,3-trifluoropropyl radicals.

Exemplary organic solvents suitable for performing step (a) of the process, include solvents chosen from ketones, for instance acetone, and cyclohexanone.

Step (b) can be carried out with stirring, optionally under an inert atmosphere.

The composition can comprise, as a relative percentage by weight of the composition, from 0.1% to 20% of grafted polysaccharides, such as from 0.5% to 10% of the grafted polysaccharide.

The composition can comprise, as a relative percentage by weight of the composition, from 5% to 90% of dimethyl ether, such as from 10% to 50% of dimethyl ether.

The cosmetically acceptable medium can comprise at least one solvent chosen from water and cosmetically acceptable solvents, such as alcohols and water-solvent mixtures. Exemplary solvents include C₁-C₄ alcohols, such as ethanol.

The composition of the invention may also contain at least one additive chosen from anionic, cationic, nonionic and amphoteric surfactants; fragrances; screening agents; preserving agents; proteins; vitamins; ceramides; polymers other than the at least one polymer; and plant, mineral and synthetic oils, and any other additive conventionally used in cosmetic compositions.

A person skilled in the art can take care to select the optional compound(s) to be added to the composition according to the invention such that the advantageous properties intrinsically associated with the composition in accordance with the invention are not adversely affected by the envisaged addition.

The present invention also relates to an aerosol device containing the composition described herein. The device can be any conventional dispenser for aerosol compositions, such as a pump dispenser, and can include components chosen from a valve, an outlet, a push button, a tube, and other components conventionally used in dispensing aerosols.

The compositions in accordance with the invention may be suitable for wet or dry hair, as styling products.

The compositions in accordance with the invention may be applied to the skin, the nails, the lips, the hair, the eyebrows and the eyelashes.

The invention will be illustrated more fully with the aid of the non-limiting example, which follows.

All the percentages are relative percentages by weight relative to the total weight of the composition, and a.m. means active material.

### EXAMPLE

An aerosol composition in accordance with the invention is prepared from the ingredients listed below:

| Cellulose containing silicone grafts [1] | 3% am |
|---|---|
| Water | 17% |
| Ethanol | 45% |
| Dimethyl ether | 35% |

[1] Hydroxypropylmethylcellulose acetate succinate containing polydimethylsiloxane grafts, as synthesized in Example No. 2 of U.S. Pat. No. 6,066,727 by the company Shin Etsu, the disclosure of which is specifically incorporated by reference herein.

[1] Hydroxypropylmethylcellulose acetate succinate containing polydimethylsiloxane grafts, as synthesized in Example No. 2 of U.S. Pat. No. 6,066,727 by the company Shin Etsu, the disclosure of which is specifically incorporated by reference herein.

This composition is sprayed onto European chestnut-brown hair. The hair shows good cosmetic properties in both cases, after drying, and shows little powdering.

What is claimed is:

1. An aerosol composition comprising:
(a) a liquid phase comprising at least one fixing material in a suitable solvent, the at least one fixing material comprising at least one polymer comprising a polysaccharide skeleton grafted with at least one group comprising at least one polysiloxane, and
(b) a propellant comprising dimethyl ether.

2. The aerosol composition according to claim 1, wherein the at least one group comprising at least one polysiloxane is grafted to at least one end of the polysaccharide skeleton.

3. The composition according to claim 1, wherein the composition comprises, as a relative percentage by weight of the composition, from 0.1% to 20% of the at least one polymer.

4. The composition according to claim 3, wherein the composition comprises, as a relative percentage by weight of the composition, from 0.5% to 10% of the at least one polymer.

5. The composition according to claim 1, wherein the composition comprises, as a relative percentage by weight of the composition, from 5% to 90% of dimethyl ether.

6. The composition according to claim 1, wherein the composition comprises, as a relative percentage by weight of the composition, from 10% to 50% of dimethyl ether.

7. The composition according to claim 1, wherein the composition further comprises at least one additive chosen from: anionic, cationic, nonionic and amphoteric surfactants; fragrances; screening agents; preserving agents; proteins; vitamins; ceramides; polymers other than said at least one polymer; and plant, mineral and synthetic oils; and any other additive conventionally used in cosmetic compositions.

8. The composition according to claim 7, wherein said at least one additive is chosen from anionic, amphoteric and nonionic fixing polymers other than said at least one polymer.

9. A method of preparing an aerosol composition, comprising:
dissolving, in an organic solvent, (a) a polysaccharide containing carboxyl groups, and (b) a polysiloxane containing an epoxy end group and corresponding to formula (I), for a time and under conditions sufficient to obtain a polysaccharide and polysiloxane mixture: 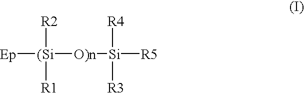
in which:
n is an integer between 3 and 500 inclusive,
R₁, R₂, R₃, R₄ and R₅ are chosen, independently of each other, from monovalent C₁, to C₁₀ hydrocarbons and monovalent C₁, to C₁₀ halohydrocarbons, and
Ep is 2-(3,4-epoxycyclohexyl )ethyl;
heating the mixture to a temperature of between 60 and 200° C. inclusive;
reacting the carboxyl groups of the polysaccharide with the epoxy groups of the polysiloxane for a time and under conditions sufficient to form a polymer comprising a polysaccharide skeleton grafted with at least one group comprising at least one polysiloxane; and
adding a propellant comprising dimethyl ether to the at least one polymer to prepare said composition.

10. The method according to claim 9, wherein the polysaccharide (a) is chosen from carboxylic, benzoyl and succinoyl groups.

11. The method according to claim 10, wherein the polysaccharide (a) is chosen from hydroxypropylmethylcellulose phthalate, hydroxypropylmethylcellulose acetate succinate, carboxymethylethylcellulose, and pullulan acetate phthalate.

12. The method according to claim 9, wherein R₁, R₂, R₃, R₄ and R₅ are chosen, independently of each other, from methyl, ethyl, propyl, butyl, C₃-C₈ cycloalkyls, aryl, C₃-C₈ aralkyls, and alkenyl groups.

13. A cosmetic hair process for fixing and/or holding a hairstyle, comprising:
applying to hair, an effective amount of a composition comprising, in a cosmetically acceptable medium:
(a) at least one polymer comprising a polysaccharide skeleton grafted with at least one group comprising at least one polysiloxane; and
(b) a propellant comprising dimethyl ether.

14. A method for holding and/or shaping a hairstyle, comprising:
applying to the hair, an effective amount of a composition comprising, in a cosmetically acceptable medium:
(a) at least one polymer comprising a polysaccharide skeleton grafted with at least one group comprising at least one polysiloxane; and
(b) a propellant comprising dimethyl ether.

15. An aerosol device comprising a container containing an aerosol composition, the composition comprising:
(a) a liquid phase comprising at least one fixing material in a suitable solvent, the at least one fixing material comprising at least one polymer comprising a polysaccharide skeleton grafted with at least one group comprising at least one polysiloxane, and
(b) a propellant comprising dimethyl ether.
